(19) United States
(12) Patent Application Publication
(10) Pub. No.: US 2020/0009361 A1
(43) Pub. Date: Jan. 9, 2020

(54) MICRO NEEDLE

(71) Applicant: GL.company, Cheongju-si (KR)

(72) Inventors: Seong Ok KIM, Cheongju-si (KR); Yong Sook YOUN, Cheongju-si (KR); So Min KIM, Cheongju-si (KR); Jae Heon KIM, Cheongju-si (KR)

(73) Assignee: GL.company, Cheongju-si (KR)

(21) Appl. No.: 16/117,999

(22) Filed: Aug. 30, 2018

(30) Foreign Application Priority Data
Jul. 4, 2018 (KR) 10-2018-0077388

(51) Int. Cl. A61M 37/00

(52) U.S. Cl. CPC A61M 37/0015; A61M 2037/0023; A61M 2207/10; A61M 2037/0053; A61M 2037/003

(57) ABSTRACT

A micro needle includes: a first base; a second base formed integrally with the top surface of the first base; a needle support formed integrally with the top surface of the second base and leading to a tapered groove penetrating the bottom surface of the first base; and a needle body formed integrally with the center of the top surface of the needle support and having a needle hole leading to the tapered groove. The micro needle allows for low-cost mass production because of the simple structure, and involves applying various drugs by injecting drugs after puncturing the skin.

### CROSS-REFERENCE TO RELATED APPLICATION

This U.S. non-provisional patent application claims priority under 35 U.S.C. § 119 of Korean Patent Application No. 10-2018-0077388, filed on Jul. 4, 2018, the entire contents of which are hereby incorporated by reference.

### BACKGROUND OF THE INVENTION

### Field of the Invention

The present invention relates to a micro needle.

### Related Art

Korean Registered Patent No. 1,719,319 (registered on Mar. 17, 2017 titled “A micro-needle structure for efficient puncturing of the skin) discloses a dissolvable micro needle that has one or more of the following characteristics: a micro needle with a tip diameter of 10 μm or smaller; a micro needle with an aspect ratio of 15:3; and a micro needle with a compressive yield strength of 10 kN/cm² or higher.

The micro needle according to the conventional art can be easily inserted into the skin without any change in shape, like bending or breaking, due to their proper mechanical strength, viscosity, and hardness, allows for effective puncturing of the skin, and shows excellent penetration of target materials into the skin as the micro needle according to the present invention dissolves. Thus, it is claimed that there is no need to press the micro needle with high force and target materials contained in any part of the micro needle can be effectively delivered into the skin. However, there are fundamental problems, including that the micro needle is difficult to mass-producing because it cannot be easily manufactured and target materials are limited because the micro needle requires solubility.

### SUMMARY OF THE INVENTION

The present invention provides a micro needle that is simple in structure, can be mass-produced, and allows for easy injection of target drugs.

A micro needle according to the present invention includes: a first base; a second base formed integrally with the top surface of the first base; a needle support formed integrally with the top surface of the second base and leading to a tapered groove penetrating the bottom surface of the first base; and a needle body formed integrally with the center of the top surface of the needle support and having a needle hole leading to the tapered groove.

Preferably, the first and second bases are disc-shaped, and the needle support is cone-shaped.

Preferably, the needle support is 1.3 to 1.7 mm long, the maximum inner diameter of the tapered groove is 1.5 to 2.5 times the outer diameter of the needle support, and the top inner diameter of the tapered groove is 1.5 to 3 times the outer diameter of the needle body.

Preferably, the needle body is 0.1 to 1.0 mm long, with an outer diameter of 40 to 300 μm, and the needle hole has an inner diameter of 30 to 250 μm.

Preferably, the needle hole is punched from the bottom of the needle support using a micro drill.

As described above, the present invention allows for low-cost mass production because of the simple structure, involves applying various drugs by injecting drugs after puncturing the skin, and causes little pain when puncturing the skin.

### BRIEF DESCRIPTION OF THE DRAWINGS

FIG. 1 is a perspective view of a micro needle according to the present invention;

FIG. 2 is a top plan view of FIG. 1;

FIG. 3 is a cross-sectional view taken along the line ‘3-3’ in FIG. 2.

### DESCRIPTION OF EXEMPLARY EMBODIMENTS

Hereinafter, an exemplary embodiment of the present invention will be described with reference to the drawings.

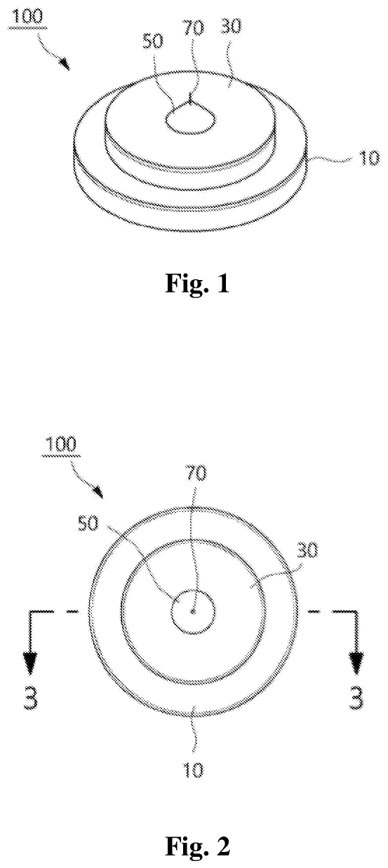

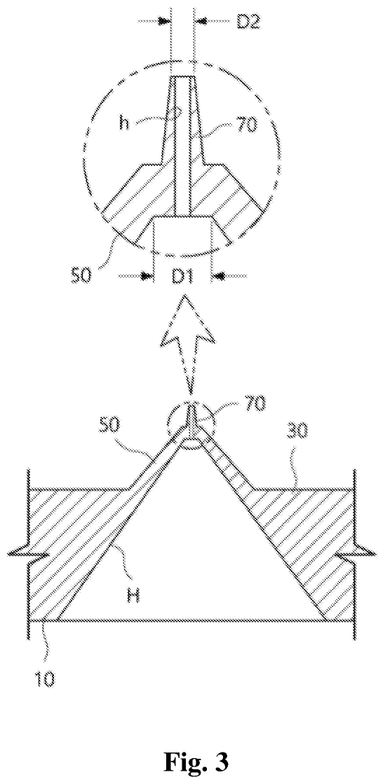

Referring to FIGS. 1 through 4, a micro needle 100 according to the present invention includes a first base 10, a second base 30, a needle support 50, and a needle body 70.

The first base 10 is disc-shaped.

The second base 30 is disc-shaped and formed integrally with the top surface of the first base 10.

Here, it should be noted that, although the first and second bases 10 and 30 are described and illustrated as being disc-shaped but are not limited to this shape and may be modified into rectangles, pentagons, or various other shapes that are uniform in thickness.

The needle support 50 is cone-shaped and formed integrally with the top surface of the second base 30, and leads to a tapered groove H penetrating the bottom surface of the first base 10.

The needle support 50 is formed into a cone shape so that it presses the surroundings of an area of skin punctured by the needle body 70 in dermatological treatments to help facilitate the puncturing, and the needle support 50 is preferably 1.3 to 1.7 mm long. Here, if the needle support 50 exceeds 1.8 mm in length, it may cause pain in dermatological treatments and, at the same time, the needle body 70 may bend or break.

Here, the tapered groove H does not penetrate the top surface of the needle support 50.

Moreover, the maximum inner diameter of the tapered groove H is 1.5 to 2.5 times the outer diameter of the needle support 50 in order to facilitate drug injection.

The needle body 70 is formed integrally with the center of the top surface of the needle support 50, and has a needle hole h that leads to the tapered groove H.

Here, the needle body 70 is preferably 0.1 to 1.0 mm long, with an outer diameter of 40 to 300 μm, and the needle hole h has an inner diameter of 30 to 250 μm. Moreover, the needle hole h should be punched from the bottom of the needle support 50 using a micro drill with a diameter of 30 to 250 μm because the needle hole h cannot be formed by injection molding.

Further, the top inner diameter D1 of the tapered groove H is 1.5 to 3 times the outer diameter D2 of the needle body 70 in order to facilitate punching of the needle hole h.

Although it is preferable that the micro needle 100 according to this embodiment is manufactured using, but not limited to, synthetic resins, it may be manufactured using non-ferrous metals.

In addition, this embodiment provides a two-stage configuration with the first and second bases 10 and 30 in order to make injection molding easy and easily mount the micro needle 100 on a micro-needle actuator (or device) later, and the micro needle 100 may have three or more stages.

Although the present invention has been described with reference to one embodiment, the present invention is not limited thereto and it is apparent that the embodiment of the present invention may be changed and modified without departing from the technical spirit of the present invention and the equivalent scopes of the claims that will be described below.

What is claimed is:

1. A micro needle comprising:
a first base;
a second base formed integrally with the top surface of the first base;
a needle support formed integrally with the top surface of the second base and leading to a tapered groove penetrating the bottom surface of the first base; and
a needle body formed integrally with the center of the top surface of the needle support and having a needle hole leading to the tapered groove.

2. The micro needle of claim 1, wherein the first and second bases are disc-shaped, and the needle support is cone-shaped.

3. The micro needle of claim 1, wherein the needle support is 1.3 to 1.7 mm long, the maximum inner diameter of the tapered groove is 1.5 to 2.5 times the outer diameter of the needle support, and the top inner diameter of the tapered groove is 1.5 to 3 times the outer diameter of the needle body.

4. The micro needle of claim 1, wherein the needle body is 0.1 to 1.0 mm long, with an outer diameter of 40 to 300 μm, and the needle hole has an inner diameter of 30 to 250 μm.

5. The micro needle of claim 1, wherein the needle hole is punched from the bottom of the needle support using a micro drill.
